(19) United States
(12) Patent Application Publication
(10) Pub. No.: US 2014/0072987 A1
(43) Pub. Date: Mar. 13, 2014

(54) METHODS OF DETECING BLADDER CANCER

(71) Applicant: RANDOX LABORATORIES LTD., Crumlin (GB)

(72) Inventors: Mark W. Ruddock, Crumlin (GB); Cherith N. Reid, Crumlin (GB); Kathleen Williamson, Belfast (GB); John V. Lamont, Crumlin (GB); Stephen P. Fitzgerald, Crumlin (GB)

(73) Assignee: RANDOX LABORATORIES LTD., Crumlin (GB)

(21) Appl. No.: 14/025,623

(22) Filed: Sep. 12, 2013

Related U.S. Application Data
(60) Provisional application No. 61/700,263, filed on Sep. 12, 2012.

(51) Int. Cl. G01N 33/68

(52) U.S. Cl. CPC G01N 33/6893
USPC 435/7.92; 435/287.2

(57) ABSTRACT

The disclosure relates to methods of detecting bladder cancer including assaying a patient sample for the levels of certain combinations of biomarkers. The disclosure also relates to methods for determining the efficacy of a drug for the treatment of bladder cancer.

### RELATED APPLICATIONS

This application claims the benefit of U.S. Provisional Application No. 61/700,263, filed Sep. 12, 2012, the contents of which are incorporated herein by reference.

### FIELD OF THE DISCLOSURE

The disclosure relates to a method of detecting the presence of bladder cancer in a patient.

### BACKGROUND

Bladder cancer is a leading cause of death worldwide. Bladder cancer is more than three times more common in men than women though the mortality rate in the latter is twice as great. Most of the patients who present with superficial bladder cancer tumours will experience a recurrence within 5 years and almost 90% of these patients will have a recurrence within 15 years. As such, it is vital that these patients are followed up on a regular basis to ensure that the cancer does not spread beyond the bladder. The constant monitoring and the costly diagnostic techniques results in bladder cancer being, on a cost per patient basis, the most expensive cancer to manage from diagnosis to death.

The usefulness of a diagnostic test is measured by its sensitivity and specificity. The sensitivity of a test is the number of true positives (the number of individuals with a particular disease who test positive for the disease) and the specificity is the number of true negatives (the number of individuals without a disease who test negative for the disease). The most common sign of bladder cancer is gross or microscopic haematuria, often detected by the family physician, and is observed in 85% of all bladder cancer patients. A simple urine dip test can be used to detect the presence of blood. Although cancer without blood is rare, leading to high sensitivity of a simple blood dip test, the specificity of the test is poor with fewer than 5% of patients presenting with haematuria actually having bladder cancer. However, the 5% of patients who do present are normally diagnosed with superficial tumours, which can easily be resected.

Cystoscopy and cytology are the preferred methods used to diagnose bladder cancer. A cytological examination involves the examination of urothelial cells in voided urine. This method has high specificity and it is convenient to obtain a sample. However, it has poor sensitivity and is subjective at low cellular yield. Cystoscopy allows direct observation of the bladder and biopsy of suspicious regions and results in 95% accuracy in diagnosis. It is therefore considered the gold standard in accurately diagnosing bladder cancer.

However, there are some disadvantages associated with cystoscopy, namely that it is extremely expensive, causes patient discomfort and does not allow for upper tract visualisation or for the detection of small areas of carcinoma in situ.

Attempts have been made in the art to identify one or more biochemical bladder cancer biomarkers that could identify patients who present with bladder cancer before committing them to cystoscopy. At the present time approximately 20% of patients present with advanced disease and their prognosis is poorer as a result. Attempts have therefore been made in the art to identify a proven marker or panel of markers, which could be used as a screening tool for bladder cancer for high-risk asymptomatic patients.

No single biomarker or panel of biomarkers has yet achieved the levels of sensitivity and specificity required to reduce the frequency of cystoscopy needed for an accurate diagnosis. NMP22 and BTA have FDA approval as point of care assays. Over the last 10 years a large number of bladder cancer markers including BTA, STAT NMP22, telomerase and FDP, have been evaluated against the gold standard urine cytology with quite consistent results of low specificity when identified singularly as biomarkers for bladder cancer. One of the reasons for low specificity is that these markers are present in urine in a large proportion of patients with urological pathologies other than bladder cancer and in patients with urinary infections.

New putative markers, such as survivin, hyaluronic acid, cytokeratin 8 and 18 and EGF, which have been shown to induce expression of the matrix metalloproteinase (MMP9) in some bladder cancer cells have been proposed as bladder cancer markers. However, none of the putative biomarkers have achieved the high specificity of urine cytology together with the high sensitivity of the telomerase assay.

Thus, in the field of bladder cancer diagnosis and treatment, the biomarkers identified in the prior art are unsatisfactory since they lack the required sensitivity and specificity necessary to make an accurate diagnosis of bladder cancer or assessment of a patient's risk in developing the disease. As a result the clinician is not able to assess accurately whether a patient should be put forward for further cytoscopic and cytological tests, which results in high costs associated with diagnosing and managing the disease. An aim of the present disclosure is to overcome these problems.

### SUMMARY

The present disclosure identifies combinations of biomarkers that can be used to either diagnose bladder cancer as an adjunct to relevant clinical parameters by replacing cystoscopy or to diagnose bladder cancer as a self-contained test. The present disclosure therefore describes combinations of biomarkers which can be used in the diagnosis of bladder cancer in a patient.

Thus, in a first aspect, the present disclosure comprises a method for determining whether a patient has bladder cancer comprising performing an assay on a sample isolated from the patient to determine the level of a combination of biomarkers selected from (i) BTA, CEA and TM and (ii) NMP22 and EGF, in the sample, wherein an increase in the level of BTA, CEA and TM or an increase in the level of NMP22 and EGF compared to a control value indicates the patient has bladder cancer.

According to a second aspect, there are described herein methods for the detection of bladder cancer in a patient comprising: obtaining at least one sample from the patient; assaying the sample from the patient for the levels of at least one combination of i) BTA, CEA and TM, and the combination of ii) NMP22 and EGF, wherein the levels of the at least one combination of biomarkers is assayed by contacting the sample with a substrate having at least one antibody against each of the biomarkers included in the at least one combination of biomarkers; providing the results of the assay for the levels of the at least one combination of biomarkers; wherein an increase in levels of the biomarkers in combination i) or an increase in the level of NMP22 and a decrease in the level of EGF in combination ii) of biomarkers compared to a control value indicates bladder cancer in the patient.

In a third aspect, the present disclosure includes a solid state device comprising a substrate having an antibody to one or more of the biomarkers selected from CEA, BTA, TM, NMP22 and EGF.

In a fourth aspect, the present disclosure includes methods for determining the efficacy of a drug for treatment of bladder cancer comprising: obtaining at least one sample from a patient treated with the drug; assaying the sample from the patient for levels of at least one combination of biomarkers selected from the combination of i) BTA, CEA and TM, and the combination of ii) NMP22 and EGF, wherein the levels of the at least one combination of biomarkers is assayed by contacting the sample from the treated patient with a solid state device comprising a substrate having at least one antibody against each of the biomarkers included in the at least one combination of biomarkers; comparing the levels of the at least one combination of biomarkers in the sample from the treated patient with levels of the at least one combination of biomarkers in a sample from an untreated patient; providing the results of the comparison of the levels of the at least one combination of biomarkers in the sample from the treated patient with the levels of the at least one combination of biomarkers in the sample from an untreated patient; wherein a decrease in the levels of the biomarkers in combination i) or a decrease in the level of NMP22 and an increase in the level of EGF in combination ii), in the sample from the treated patient compared with the levels of the at least one combination of biomarkers in the sample from an untreated patient indicates that the drug has efficacy as a treatment for bladder cancer.

### DESCRIPTION OF THE DRAWINGS

FIGS. 1A and 1B show predicted probabilities of 4 algorithms. Each algorithm, created using Forward Wald binary logistic regression analyses, generated a predicted probability between 0 and 1 for each patient (represented by a circle). For (CON) controls predicted probabilities <0.5, that is, below the 0.5 predicted probability line indicate correctly classified cases. Conversely, for urothelial cancers, correctly classified cases appear above this line. Predicted probabilities were generated for each patient using 4 algorithms according to their diagnostic classification as (A) (shown in FIG. 1A): ND, no diagnosis; benign, benign pathologies; INF, inflammatory conditions BPH, benign prostrate hyperplasia cancers, cancers other than urothelial cancer; Sup, superficial Ur Ca; Inv, invasive Ur Ca and (B) (shown in FIG. 1B): as CON, NEW, newly diagnosed, or (RECUR), recurrence; PPP, prior predicted probability; VEGF, vascular endothelial growth factor; AUC, area under the curve.

FIG. 2 depicts Table 1, which shows the characteristics of the patients investigated in an analysis.

FIG. 3 depicts Table 2, which shows the analyses of significant differences in biomarker profiles of samples obtained from bladder cancer patients and controls.

FIG. 4, depicts Table 3, which shows biomarker sensitivities and specificities as determined by the effect of the presence or absence of a particular biomarker on a combination of biomarkers.

### DESCRIPTION

The present disclosure is based on the finding that the level of specific biomarker combinations in blood and/or urine samples isolated from a patient who has bladder cancer is significantly different to that in controls. The identification of such biomarker combinations enable an accurate diagnosis of bladder cancer to be made. This is advantageous since it decreases the need for invasive diagnostic procedures.

In the context of the present disclosure the term “bladder cancer” is understood to include urothelial carcinoma, bladder squamous cell carcinoma or bladder adenocarcinoma. Preferably, the cancer with which the present disclosure is concerned is urothelial carcinoma.

In the context of the present disclosure, a “control” or “control value” is understood to mean the level of a particular biomarker typically found in patients who do not have bladder cancer. The control level of a biomarker may be determined by analysis of a sample isolated from a person with haematuria but who does not have bladder cancer or may be the level of the biomarker understood by the skilled person to be typical for such a person. The control value of a biomarker may be determined by methods known in the art and normal values for a biomarker may be referenced from the literature from the manufacturer of an assay used to determine the biomarker level.

The “level” of a combination of biomarkers refers to the amount, expression level or concentration of each biomarker of the combination of biomarkers within the sample.

A number of biomarkers present in a sample isolated from a patient having bladder cancer may have levels which are different to that of a control. However, the levels of some of the biomarkers that are different compared to a control may not show a strong enough correlation with bladder cancer such that they may be used to diagnose bladder cancer with an acceptable accuracy. Accuracy of a diagnostic method is best described by its receiver-operating characteristics (ROC) (Zweig, M. H., and Campbell, G., Clin. Chem. 39 (1993) 561-577). The ROC graph is a plot of all of the sensitivity/specificity pairs resulting from continuously varying the decision threshold over the entire range of data observed. The combinations of biomarkers used to diagnose bladder cancer in the present disclosure have a sensitivity and specificity of at least 70%. This means that out of 100 patients which have bladder cancer, 70% of them will be correctly identified from the determination of the presence of a particular combination of biomarkers as positive for bladder cancer while out of 100 patients who do not have bladder cancer 70% will accurately test negative for the disease.

A ROC plot depicts the overlap between the two distributions by plotting the sensitivity versus 1—specificity for the complete range of decision thresholds. On the y-axis is sensitivity, or the true-positive fraction defined as [(number of true-positive test results)/(number of true-positive+number of false-negative test results)]. This has also been referred to as positivity in the presence of a disease or condition. It is calculated solely from the affected subgroup. On the x-axis is the false-positive fraction, or 1—specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. Qualitatively, the closer the plot is to the upper left corner, the higher the overall accuracy of the test.

One convenient goal to quantify the diagnostic accuracy of a laboratory test is to express its performance by a single number. The most common global measure is the area under the curve (AUC) of the ROC plot. The area under the ROC curve is a measure of the probability that the perceived measurement will allow correct identification of a condition. By convention, this area is always 0.5. Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0). In the context of the present disclosure, the two different conditions are whether a patient has or does not have bladder cancer.

The combinations of biomarkers identified by the present inventors as being useful to detect bladder cancer in patients are (i) BTA, CEA and TM and (ii) NMP22 and EGF. These biomarker combinations were identified by statistical analysis based on a diagnostic algorithm of demographic variables which may include one or more of the patient's age, whether he smokes and the number of smoking years and whether he takes anti-hypertensive medication. Each of these demographic variables may be assigned a notional value which is used in Forward Wald binary logistic regression analyses to create a diagnostic algorithm designated as PPP. PPP represents, in a single measure, the intrinsic contribution toward group membership with which each subject commences screening. The contribution that each biomarker makes to the area under the curve (AUC) values for the PPP algorithm is assessed to determine whether a combination of biomarkers increases the statistical significance of the PPP algorithm.

The biomarkers in combination i) are all present at an increased level in bladder cancer patients compared to a control. In combination ii), NMP22 is increased, whereas EGF is decreased compared to a control.

In one aspect of the present disclosure, the patient to be tested for the bladder cancer presents with haematuria. Haematuria may be caused by a number of conditions, such as bladder cancer, prostate cancer or urinary tract infections. The identification of the combinations of the biomarkers used in the present disclosure in samples isolated from the patient allows confirmation of bladder cancer to be made in patients with haematuria. Thus, in the clinical setting, the clinician may make an assessment of the patient's medical history and note the patient's age and whether he smokes and, if so, for how long. The variables may be assigned notional values and fed into regression analysis software, such as Forward Wald binary regression analysis software, which is known in the art. The level of the biomarkers present in the sample isolated from the patient is then determined and these values are also fed into the regression analysis. An AUC value is generated from the regression analysis of between 0 and 1. Values greater than 0.6 indicate that the patient has bladder cancer, whilst values less than 0.4 indicate that the patient does not have cancer. Values of 0.4 to 0.6 indicate that the analysis has been inconclusive and that further evaluation of the patient is required.

The biomarkers are detected in at least one sample that is isolated from the patient. The sample may be a urine sample, blood sample, serum sample or plasma sample. Preferably the levels of the biomarkers present in the combinations under investigation may be determined in a urine sample or a blood sample. The CEA biomarker is detected in a blood sample whereas the other biomarkers are detected in urine.

The methods of the disclosure may be carried out using a substrate having at least one antibody against each of the biomarkers included in the at least one combination of biomarkers.

The antibodies that may be used in the present disclosure can be of any conventional type. Polyclonal and monoclonal antibodies are preferred, with monoclonal antibodies being most preferred.

In one embodiment, the substrate is a multiwell microtitre plate, for use in an ELISA method. Accordingly, the determination of the level of the biomarkers in the sample may be determined by commercially available methods such as an ELISA based assay, chemical or enzymatic protein determination. Preferably, the methods of the present disclosure use a solid state device for determining the level of the biomarkers in the sample isolated from the patient. The solid state device comprises a substrate having an activated surface on to which an antibody to the biomarker of interest is immobilised at discreet areas of the activated surface. Preferably, the solid state device may perform multi-analyte assays such that the level of a biomarker of interest in a sample isolated from the patient may be determined simultaneously with the level of a further biomarker of interest in the sample. In this embodiment, the solid state device has a multiplicity of discrete reaction sites each bearing a desired antibody covalently bound to the substrate, and in which the surface of the substrate between the reaction sites is inert with respect to the target biomarker. The solid state, multi-analyte device may therefore exhibit little or no non-specific binding.

A device that may be used in the disclosure may be prepared by activating the surface of a suitable substrate, and applying an array of antibodies on to discrete sites on the surface. If desired, the other active areas may be blocked. The ligands may be bound to the substrate via, a linker. In particular, it is preferred that the activated surface is reacted successively with an organosilane, a bifunctional linker and the antibody. The solid state device used in the methods of the present disclosure may be manufactured according to the method disclosed in, for example, GB-A-2324866 the content of which is incorporated herein in its entirety. Preferably, the solid state device used in the methods of the present disclosure is the Biochip Array Technology system (BAT) (available from Randox Laboratories Limited). More preferably, the Evidence Evolution and Evidence Investigator apparatus (available from Randox Laboratories) may be used to determine the levels of biomarkers in the sample.

The methods of the present disclosure maybe carried out as follows:

A number of risk factors are known in the bladder cancer development. Age and smoking are the most accurate discriminating factors in determining whether a patient who presents with haematuria has bladder cancer or some other pathology. Therefore, in order that the combinations of biomarkers provide an accurate means of detecting the presence or risk of bladder cancer as opposed to another pathology which clinically presented as haematuria, known risk factors can be assessed in each patient and the effect on the presence of each of the proposed biomarker combinations taken in to account. Thus, a patient can be assessed for their exposure to various bladder cancer risk factors by answering a questionnaire, asking, for example, the patient's age and sex and whether there is a family history of bladder cancer. Other risk factors can be investigated, including whether the patient suffers from renal stone disease, recurrent urinary infections, benign prostatic hypertrophy or malignant diseases and whether he has received pelvic radiotherapy. It is desirable to establish whether the patient is a smoker and, if so, the length of time as a smoker and the quantity and type of tobacco smoked (pipe or cigarette), his alcohol consumption and medical history. The patient's medical history is of particular significant since a number of drugs are known to affect the expression of a number of the biomarkers in the patient's blood and/or urine. Drugs which have such an effect may be selected from anti-hypertensive drugs, anti-cholesterol drugs, antiplatelets drugs, anti-ulcer drugs, prostate reduction drugs, anti-asthma drugs, analgesic drugs, anti-depressant drugs, anti-inflammatory drugs, anti-diabetes drugs, anti-coagulant drugs, anti-anxiety drugs and vitamins.

The risk factors positively identified can be assigned a starting predictive probability (SPP) which is an indicator of each risk factor's contribution to the development of bladder cancer in the patient. The SPP is based on the average value of each risk factor for a patient presenting with bladder cancer e.g. average age and average number of cigarettes smoked. As a result, the statistical analysis conducted on the various biomarker combinations can take into account the possible effect that each risk factor may have on the development of bladder cancer and the presence of a particular biomarker.

Urine samples (50 ml) and serum samples (2 ml) can be collected from the patient in sterile containers. Unfiltered and uncentrifuged urine samples can be immediately aliquoted and stored at −80° C. until analyses. Urine samples can be thawed on ice and then centrifuged (1200×g, 10 minutes, 4° C.) to remove any particulate matter prior to analysis. Preparations can be stained with Papanicolaou and Geimsa prior centrifugation, to indicate samples as either insufficient for analysis, normal, atypical, suspicious or malignant. The presence of inflammatory cells can also be recorded.

In a preferred embodiment, the biomarker assay is carried out using a solid state device. For example, the Randox Biochip Array Technology (BAT) can be used to detect the presence of the various biomarkers. Following antibody activation with assay buffer, standards and samples can be added and incubated at 37° C. for 60 minutes, then placed in a thermo-shaker at 370 rpm for 60 minutes. Antibody conjugates (HRP) can be added and incubated in the thermo-shaker at 370 rpm for 60 minutes. The chemiluminescent signals formed after the addition of luminol (1:1 ratio with conjugate) can then be detected and measured using digital imaging technology and compared with that from a calibration curve to calculate concentration of the analytes in the samples. The analytical sensitivity of the biochip is as follows:

| | | Preferred | Most preferred |
|---|---|---|---|
| | Range | range | range |
| Combination i) | | | |
| BTA (U/mL) | 10-400 | 15-370 | 40-100 |
| CEA | 1-4 | 1.2-3 | 1.5-2.5 |
| (Serum)(ng/mL) | | | |
| TM (ng/mL) | 2-7 | 3-6 | 2.5-4.5 |
| Combination ii) | | | |
| EGF (pg/mL) | 2000-10000 | 3000-8000 | 4000-7000 |

| | |
|---|---|
| NMP22 | This biomarker is assessed qualitatively, with a |
| | positive or negative result (<10 U/mL being |
| | negative). |

Means of triplicate biomarker measurements for each identified biomarker can then be transformed to achieve normal distributions. Biomarker sensitivities and specificities for bladder cancer can be determined from ROC analyses.

Forward Wald binary logistic regression analysis (cut off probability for case classification=0.5) can be used and regression analysis can be conducted using SPSS regression software.

Each combination of biomarkers has a sensitivity and specificity of at least 70%. This means that out of 100 patients which have bladder cancer, 70% of them will be correctly identified from the determination of the presence of particular combination of biomarkers as positive for bladder cancer (sensitivity test) while out of 100 patient who do not have bladder cancer 70% will accurately test negative for the disease (sensitivity test). Preferably, the combination of biomarkers has a sensitivity of at least 75%. More preferably the sensitivity will be at least 80%, and the specificity will be at least 75%, more preferably of at least 80%.

The following Example, with reference to Tables 1 to 3, describes methods by which the biomarkers may be detected as having increased levels in a urine or blood sample isolated from a patient. The biomarkers which were identified were then analysed statistically in order to identify particular combinations of biomarkers which correlate with the patient having bladder cancer.

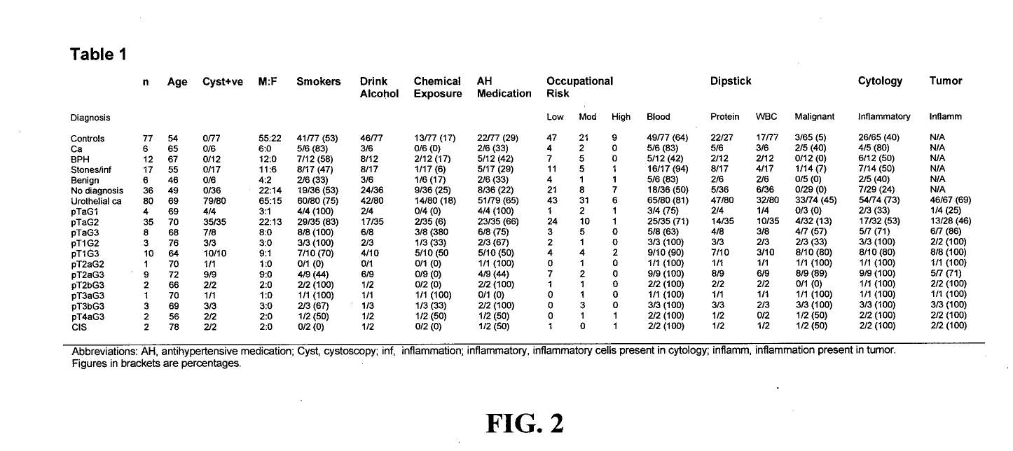

Table 1 (depicted in FIG. 2) shows the characteristics of the patients investigated in the analysis;

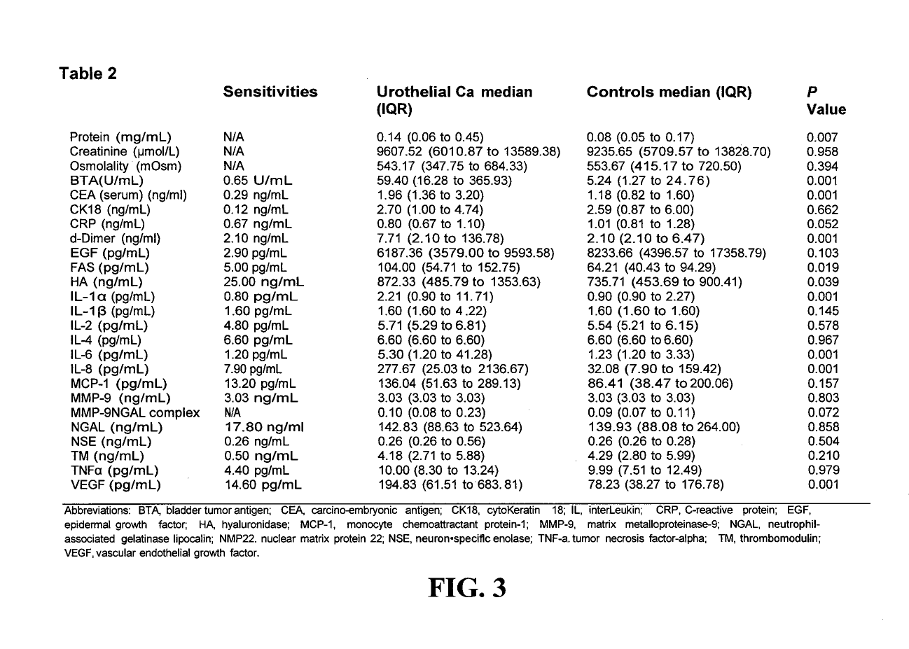

Table 2 (depicted in FIG. 3) shows the analyses of significant differences in biomarker profiles of samples obtained from bladder cancer patients and controls; and

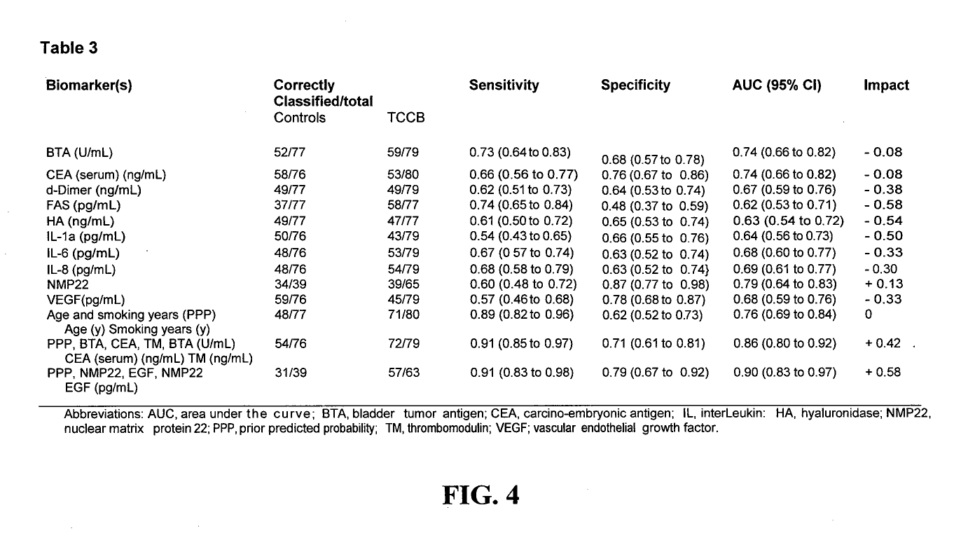

Table 3 (depicted in FIG. 4) shows biomarker sensitivities and specificities as determined by the effect of the presence or absence of a particular biomarker on a combination of biomarkers.

### EXAMPLES

A prospective case-control study to explore the contributions of demographic and clinical factors to a diagnostic algorithm were planned to determine prior predicted probability (PPP) based on the age of a patient and whether he smokes and the number of years he has done so. 23 bio-markers were appraised, representing proteins from diverse pathways involved in bladder cancer carcinogenesis.

Patients presenting with haematuria with planned cystoscopy were recruited. After written informed consent, we collected urine (50 mL) and serum (2 mL) samples from each patient. Samples were stored at −80° C. until biomarker analyses (undertaken within 12 months of collection). Aution Sticks 10EA used for dipstick analyses were interpreted using PocketChem (Arkray factory, Inc., Japan). NMP22 was assessed qualitatively (<10 U/mL negative) (Matritech Inc, Newton, Mass.). Cytology was assessed on Papanicolaou and Giemsa-stained preparations.

Clinicopathological data were recorded at the time of recruitment. Each patient's occupational history was scored as low risk (score=1), moderate risk (score=2), or high risk (score=3). Scores were averaged. Occupations classed as high risk included painters, wood lathe operators, and dye mixers. Current medications were grouped into 14 categories: antihypertensives (AH), anticholesterol, antiplatelets, antiulcer, benign prostate hyperplasia (BPH) therapy, that is, a-blocker and 5 a-reductase inhibitor, antiasthma, analgesics, antidepressants, anti-inflammatory, antidiabetes, anxiolytics, anticoagulants, and vitamins. After investigations patients were classified as “no diagnosis,”. “benign pathologies,” “stones and inflammation,” “BPH,” “other cancers,” or “urothelial cancer.”

Sixteen biomarkers in urine and 3 in serum: carcinoembryonic antigen (CEA), free prostate-specific antigen (FPSA), and total PSA (TPSA) were measured in triplicate using Randox biochip array technology (Randox Evidence and Investigator), which is a multiplex system for protein analysis (FIG. 2, Table 1). PSA analyses were undertaken for diagnostic confirmation and quality control purposes only.

BTA was measured using BTA TRAK enzyme-linked immunosorbent assays (ELISAs) from Polymedco, Inc., Cortlandt Manor, N.Y.; epidermal growth factor (EGF) and the MMP-9 NGAL complex were measured using standard ELISAs. Single measurements were carried out for hyaluronidase (HA), FAS, and cytokeratin (CK 18) using ELISAs from Echelon Biosciences Inc. (Salt Lake City, Utah), Raybio, Inc. (Norcross, Ga.), and USCNLIFE Science & Technology Co. Ltd. (China), respectively. All other biomarkers were measured in triplicate.

Creatinine levels (mol/L) and osmolarity (mOsm) were measured in triplicate using a Daytona RX Series Clinical Analyzer (Randox) and a Loser Micro-Osmometer (Type 15) (Loser Messtechnik, Germany), respectively. Total protein levels (mg/mL) in urine were determined using Bradford assay A595 nm (Hitachi U2800 spectrophotometer) and BSA as standard.

Statistical Analyses

Average measurements for each biomarker were divided by the average creatinine level measured in the same patient's urine sample and then log transformed before statistical analyses using SPSS v17.

Receiver operating characteristic (ROC) curves were created to rank area under the curves (AUCs). From these we determined the cut-off limit for each biomarker/algorithm that would delineate positive from negative test results. This point was taken as the measured level at the minimum distance from the top of the y-axis of the ROC curve, that is, the point of maximum specificity.

Demographic variables were entered into a Forward Wald binary logistic regression analyses (cut-off probability for case classification=0.5) to create a diagnostic algorithm that was designated as PPP. PPP was created because the baseline characteristics of the bladder cancers and control groups were different. PPP represents, in a single measure, the intrinsic contribution toward group membership that each subject commences with.

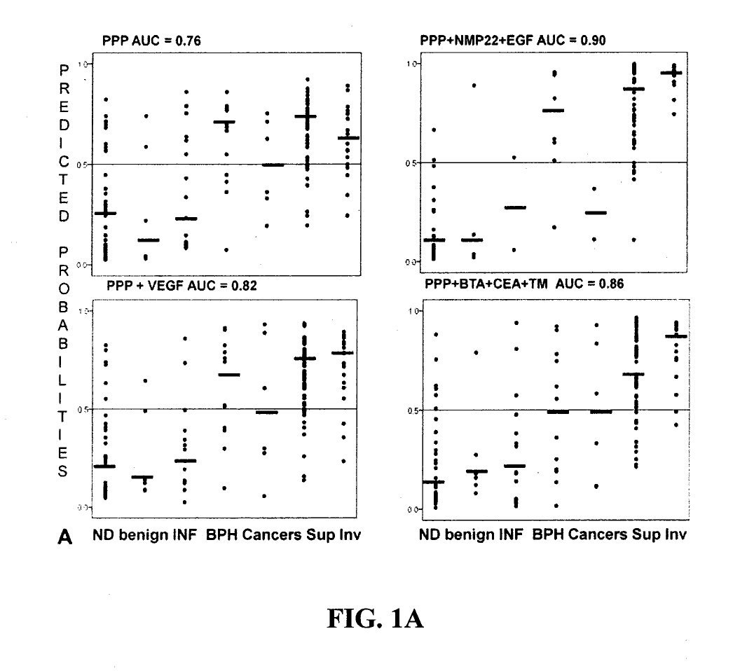

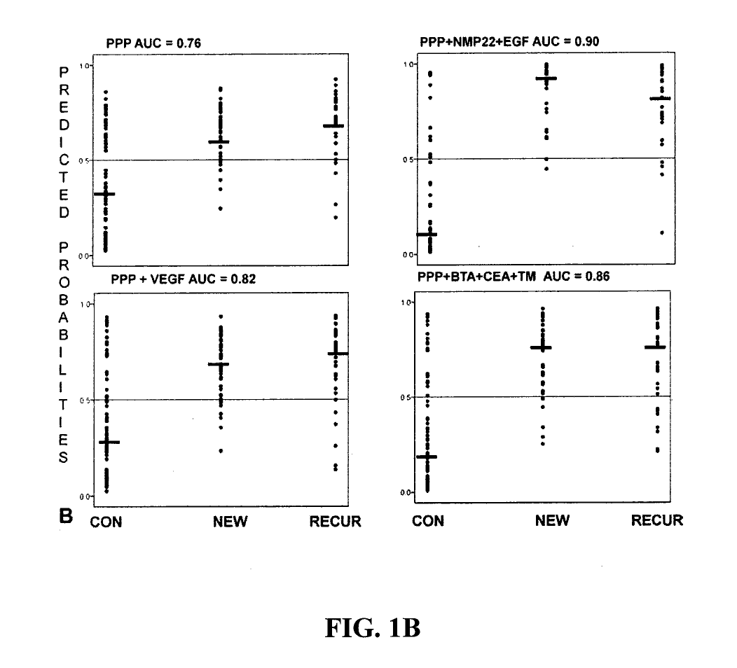

It was then determined whether addition of single biomarkers or sets of biomarkers could significantly increase the AUC of the PPP algorithm. Principle components analysis (PCA) was undertaken (rotation method: Varimax with Kaiser normalization) to reduce the dimensionality of the data and then ran a series of regression analyses entering 5 or more biomarkers for each analysis. To determine the impact of biomarkers/algorithms, that is, their additional impact over demographics (PPP) the equation (new AUC−PPP AUC)/(1−PPP AUC), was used where the new AUC is the biomarker/algorithm AUC and PPP AUC is the AUC for PPP, taking 0.6 as the threshold for a significant impact. Predicted probabilities against final disease classifications were plotted as scatter charts (FIG. 1).

Results

Eight biomarkers were significantly higher in bladder cancers compared with controls (FIG. 2, Table 1) and EGF was lower than the control. Normal distributions in frequency histograms plotted using log transformed data for all biomarkers were observed except for that of von Willebrand factor (vWF), FPSA, and TPSA. The latter two were statistically analyzed using nonparametric methods. vWF was excluded from subsequent statistical analyses. Creatinine and osmolarity levels were significantly correlated in urine (r=0.796, Pearson correlation). FPSA and TPSA levels (measured as controls) were significantly higher in males (n=119) (median=0.11; IQR=0.05-0.18, and median=0.88; IQR=0.04-2.50, respectively) ng/mL than in females (n=37) (median=0.04 (IQR=0.04-0.04) and median=0.06; IQR=0.06-0.06, respectively) ng/mL (Mann-Whitney; P<0.001).

CEA behaved favourably against BTA and NMP22 as a single biomarker for bladder cancer, contributed to 1 of the algorithms and was the most accurate predictor for BPH (83% sensitivity) (FIG. 2, Table 1, and FIG. 3, Table 2). CEA was significantly elevated in smokers (median=1.77; IQR=1.18-2.65) compared with non-smokers (median=1.15; IQR=0.76-1.80) ng/mL (P=0.003, t test). When smoking quantity, smoking years and CEA were entered into the Forward Wald binary logistic regression analysis, both smoking years and CEA were retained in the equation, indicating that CEA acts independently of smoking as a biomarker for bladder cancer.

Levels of nine biomarkers were significantly different when non-muscle invasive and muscle invasive were compared and 7 when grades 1 and 2 combined were compared with grade three tumours (t test; P<0.05) (FIG. 4, Table 3). Urinary levels of interleukin (IL)-8 were significantly higher in urines from patients with tumours with an inflammatory infiltrate (n=46) when compared with those without an inflammatory component (n=21) (P=0.015, t test). Two algorithms with enhanced AUCs in comparison to PPP were also found (FIG. 4, Table 3).

The present disclosure describes methods of identifying patients who have bladder cancer through the detection of specific combinations of biomarkers. The clinician is able to accurately determine whether a patient presenting with haematuria has bladder cancer, or some other ailment, by detecting the presence of one of the combinations of biomarkers identified by the present disclosure from the patient's blood or urine.

1. A method for the detection of bladder cancer in a patient comprising:
obtaining at least one sample from the patient;
assaying the sample from the patient for the levels of at least one combination of biomarkers selected from the combination of i) BTA, CEA and TM, and the combination of ii) NMP22 and EGF, wherein the levels of the at least one combination of biomarkers is assayed by contacting the sample with a substrate having at least one antibody against each of the biomarkers included in the at least one combination of biomarkers;
providing the results of the assay for the levels of the at least one combination of biomarkers;
wherein an increase in levels of the biomarkers in combination i) or an increase in the level of NMP22 and a decrease in the level of EGF in combination ii) compared to a control value indicates bladder cancer in the patient.

2. The method of claim 1, wherein the bladder cancer is urothelial carcinoma.

3. The method of claim 1, wherein the patient has haematuria.

4. The method of claim 1, wherein the sample is selected from the group consisting of urine, blood, plasma and serum.

5. The method of claim 1, wherein the level of CEA is determined in a blood sample and the level of BTA, TM, NMP22 and EGF is determined in a urine sample.

6. The method of claim 1, wherein the substrate is part of a solid state device.

7. The method of claim 1, wherein the substrate is a multiwell microtitre plate and the level of CEA, BTA, TM, NMP22 or EGF is determined by an ELISA based assay.

8. A solid state device comprising a substrate comprising an antibody to one or more of the biomarkers selected from CEA, BTA, TM, NMP22 and EGF.

9. The solid state device of claim 8, wherein the antibody is a monoclonal antibody.

10. A method for determining the efficacy of a drug for treatment of bladder cancer comprising:
obtaining at least one sample from a patient treated with the drug;
assaying the sample from the patient for levels of at least one combination of biomarkers selected from the combination of i) BTA, CEA and TM, and the combination of ii) NMP22 and EGF, wherein the levels of the at least one combination of biomarkers is assayed by contacting the sample from the treated patient with a solid state device comprising a substrate having at least one antibody against each of the biomarkers included in the at least one combination of biomarkers;
comparing the levels of the at least one combination of biomarkers in the sample from the treated patient with levels of the at least one combination of biomarkers in a sample from an untreated patient;
providing the results of the comparison of the levels of the at least one combination of biomarkers in the sample from the treated patient with the levels of the at least one combination of biomarkers in the sample from an untreated patient;
wherein a decrease in the levels of the biomarkers in combination i) or a decrease in the level of NMP22 and an increase in the level of EGF in combination ii) in the sample from the treated patient compared with the levels of the at least one combination of biomarkers in the sample from an untreated patient indicates that the drug has efficacy as a treatment for bladder cancer.

11. A method according to claim 10, wherein the substrate is part of a solid state device.
